(19) United States
(12) Patent Application Publication
(10) Pub. No.: US 2026/0174640 A1
(43) Pub. Date: Jun. 25, 2026

(54) SUNSCREEN FORMULATION COMPRISING MICROCAPSULES, AND PROCESS FOR MANUFACTURING SUCH MICROCAPSULES

(71) Applicant: EXOSHIELD, Carros (FR)

(72) Inventors: Bruno MONTAGNIER, Marseille (FR); Denis PECH, Vence (FR); Alexandre DINGAS, Le Cannet (FR); François MARCHIO, Paris (FR)

(73) Assignee: EXOSHIELD, Carros (FR)

(21) Appl. No.: 19/127,811

(22) PCT Filed: Nov. 8, 2023
(86) PCT No.: PCT/IB2023/061260

§ 371 Date: May 7, 2025

(30) Foreign Application Priority Data
Nov. 8, 2022 (FR) FR2211583

(51) Int. Cl. A61K 8/11; A61K 8/87; A61K 8/88; A61Q 17/04

(52) U.S. Cl. CPC A61K 8/11; A61K 8/87; A61K 8/88; A61Q 17/04; A61K 2800/624

(57) ABSTRACT

Disclosed is a core-shell microcapsule comprising a core containing at least one active ingredient, and comprising a shell, said shell constituting a wall around the core and representing a mass fraction of at least 20% of the total mass of the microcapsule, said microcapsule being characterized in that the shell comprises at least one crosslinked polymer, preferably obtained by interfacial polymerization. The size of the microcapsules is preferably less than 1 μm. The active ingredient can be a sunscreen.

### CROSS-REFERENCE TO RELATED APPLICATIONS

The present application is a National Stage Application of PCT International Application No. PCT/IB2023/061260 (filed on Nov. 8, 2023), under 35 U.S.C. § 371, which claims priority to French Patent Application No. FR 2211583 (filed on Nov. 8, 2022), which are each hereby incorporated by reference in their complete respective entireties.

### TECHNICAL FIELD

The present invention relates to the field of cosmetology, and more particularly to that of sun protection formulations or formulations providing protection against photo-aging of the skin or skin appendages, intended to be applied to the skin and comprising substances called sun filters.

These formulations can be presented in particular in the form of liquid or pasty emulsions, such as ointments, creams, milks, mousses, or in the form of solid emulsions such as sticks, bars, breads, or in non-emulsified form, such as oils, suspensions, gels, lotions, loose or compact powders.

More specifically, the invention relates to a formulation microencapsulated in a thick shell formed by a crosslinked polymer. These microcapsules are sub-micron in size.

### BACKGROUND

Sunscreen formulations or products claiming protection against photoaging of the skin and skin appendages include specific molecules capable of attenuating the ultraviolet rays (commonly abbreviated as “UV”) of the solar spectrum. This attenuation is achieved by absorption or reflection, total or partial, of at least part of the near ultraviolet spectrum by molecules called here ultraviolet filters. It has long been known that, in general, UV rays are harmful to the skin, and in particular UV rays belonging to two spectral zones designated by those skilled in the art as “UV-A” (approximately 320 nm to 400 nm) and “UV-B” (approximately 280 nm to 320 nm). UV rays with shorter wavelengths are the most aggressive, and UV rays with longer wavelengths penetrate deeper into the skin. They can cause immunosuppression and skin cancers. More specifically, UV-A rays induce immediate pigmentation of the skin, but also premature aging (photoaging). UV-B rays induce the synthesis vitamin D in the skin and delayed skin pigmentation (tanning phenomenon), but also sunburn.

The most effective UV filters protect against both UV-A and UV-B rays. There are organic UV filters and mineral UV filters.

Organic UV filters (also called chemical UV filters) are organic molecules that absorb and dissipate UV rays through chemical reactions. The majority of these organic UV filters are lipophilic. Their maximum concentrations and their combinations with each other in sunscreen formulations or products claiming protection against photo-aging of the skin and skin appendages are regulated. Examples of organic UV filters include oxybenzone, octrocrylene, avobenzone, octyl-methoxycinnamate. Organic UV filters have the advantage of being easily incorporated into user-friendly sunscreen formulations.

However, some organic UV filters have disadvantages, including photo-instability (for example, avobenzone), allergenic potential (for example, alkyl para-aminobenzoates), and many of them have a certain capacity to cross the skin barrier (stratum corneum) (for example, ethylhexyl methoxycinnamate), and may show endocrine disrupting effects (for example, oxybenzone). For example, in the United States of America, the Food & Drug Administration (FDA) now requires additional toxicological studies for organic sunscreens if these molecules are found in plasma at a concentration greater than 0.5 ng/ml.

And finally, all these molecules, despite their lipophilic nature, will end up in natural environments, where they represent chemical pollution that appears less and less acceptable. For example, two molecules commonly used as UV filters, namely benzophenone-3 and ethylhexyl methoxycinnamate, are considered toxic to aquatic environments, and some local authorities (such as the State of Hawaii) have banned or restricted the use of sunscreen formulations containing these molecules.

Mineral UV filters are typically made of insoluble inorganic powders, such as titanium dioxide, zinc oxide, cerium oxide or iron oxides. These particles reflect UV-A and UV-B rays. These powders must be dispersed. To facilitate their incorporation into sunscreen formulations, these Inorganic particles are usually coated with a hydrophilic or hydrophobic coating (e.g., based on methoxysilane, dimethicone, silica or alumina). This coating partially inhibits their photoreactivity, because these powders are metal oxides capable of releasing hydrogen peroxide (H₂O₂) into water by photocatalytic reaction. It is responsible for damage to biological material (planktonic animals).

Mineral UV filters have the advantages of being hypoallergenic and not crossing the skin barrier. On the other hand, they are more difficult to formulate than organic UV filters, because the formulations of sunscreen or products claiming protection against photo-aging of the skin and skin appendages in which these UV filters are incorporated are often thicker and tend to leave unsightly white marks on the skin. To overcome this problem, the particle size of these mineral UV filters can be reduced by making them nanometric. This increases sun protection but the regulations then impose specific marking and the prohibition of dispensing in vaporizable or aerosol form.

Formulations of sunscreen or products claiming protection against photoaging of the skin and skin appendages are typically characterized by their sun protection factor (abbreviated SPF), defined according to the following formula:

$$SPF = ( Minimum Erythema Dose on protected skin ) / ( Minimum Erythema Dose on unprotected skin )$$

Formulations of sunscreens or products claiming protection against photo-aging of the skin and skin appendages including UV filters must meet numerous requirements in order for photoprotection to be optimally effective. In particular, these formulations must have low penetration into the skin of active substances such as UV filters, to limit toxicity or allergy problems. The formulations must also have good emollient properties to allow pleasant and long-lasting application to the skin surface. It is also desirable that they are film-forming and water-resistant, but not sticky, especially since frequent reapplication is recommended during sun exposure.

According to the state of the art, sunscreen compositions or products for protection against photo-aging of the skin and skin appendages can be present in different types of formulation, among which we can cite among the forms of liquid or pasty emulsions: ointments, creams, milks, mousses, or in the form of solid emulsions: sticks, sticks, breads, or in non-emulsified form: oils, suspensions, gels, lotions, loose or compact powders. The formulation of a sunscreen product depends on the physicochemical properties of the incorporated UV filters. When the photoprotection product is an emulsion, it comprises a lipid phase, an aqueous phase and one or more surfactants. UV filters are dispersed either in the lipid phase if they are lipophilic, or in the aqueous phase if they are hydrophilic. It is known to add excipients to sunscreen formulations to ensure optimal distribution of UV filters on the skin surface by forming a homogeneous protective film upon application.

Generally speaking, it is not desirable for UV filters to penetrate the skin, as they can be toxic or allergenic. These two phenomena can be caused or reinforced by the effect of UV light which can make these filters phototoxic or photoallergenic; if these filters are photounstable, their decomposition products can also present these undesirable effects.

The passage of UV filters through the skin can be promoted in particular by a poor general condition of the skin, by the disorganization of the lipids of the skin barrier under the action of UV rays, by the presence of certain solvents such as ethanol, propylene glycol, surfactants, by the presence of certain emollient agents, alpha-hydroxy acids (commonly abbreviated AHA), and by the molecular mass of the filters, knowing that molecules with a molecular mass of less than 500 Da are more likely to cross the skin barrier than molecules with a higher molecular mass.

However, according to the state of the art, organic UV filters are often low molecular weight lipophilic molecules; they are therefore likely to pass the skin barrier and thus reach the nucleated cells of the skin, then the systemic circulation. To limit this passage through the skin barrier, the state of the art offers formulations of sun products or products claiming protection against photo-aging of the skin and skin appendages in which the UV filter is trapped in a particle. In particular, many embodiments are known in which UV filters are enclosed in microcapsules. These particles can also absorb and/or reflect the UV radiation. The outer wall of these particles can be chosen from a material that allows the integrity of the mixture of sunscreens to be maintained by sufficient sealing; it is desirable that this material be stable under solar irradiation to allow an application with a prolonged effect.

The state of the art includes many particulate systems. For example, we know of solid lipid nanoparticles (SLN). These are oily droplets of lipids that are solid at body temperature and stabilized by surfactants. In other words, SLNs are nanoparticles consisting of a solid lipid core enveloped by one or more surfactant(s) suspended in an aqueous phase; they thus possess occlusive properties which could make them usable for cosmetic sun protection products. It would thus be possible to prepare formulations containing SLNs in which liposoluble UV filters are encapsulated; with these formulations the penetration of the encapsulated UV filters into the skin would be reduced. Such compositions are described in U.S. Patent Application Publication No. 2003/0235540.

However, the use of SLNs has the following disadvantages: on the one hand, the quantities of encapsulated UV filters are limited, and on the other hand, these nano-encapsulated systems are not completely stable: the UV filters tend to be expelled from the nanoparticle during storage of the sunscreen formulation. This latter problem is inherent in the solid lipids constituting the SLN matrix which tend to form a perfect crystalline network whose interstices make it possible to eject UV filters from the nanoparticle.

Furthermore, SLNs can be more or less sensitive to heat depending on the melting point of the solid lipid matrix used. If the solid lipid matrix melts, this can disrupt the system, which may lead to a reduction in the UV filtering power of SLNs but also to the total phase shift of the SLN suspension. Finally, the significant presence of solid lipids whose melting point is higher than skin temperature (i.e., higher than approximately 32° C.) makes SLN-based sunscreen formulations difficult to spread.

Thus, given the current state of knowledge on the properties of SLNs, sunscreen formulations comprising UV filters encapsulated with this system do not prove to be fully satisfactory.

UV filter trapping systems are also known, consisting of nanoparticles made up of a solid and liquid lipid core enveloped by one or more surfactant(s), resulting from the mixing of solid lipids with liquid lipids. These particles, which can be suspended in an aqueous phase, are known as nanostructured lipid carriers (NLCs). Compared to SLNs, NLCs have a heterogeneous structure that gives their matrix an imperfect structure with spaces in which UV filters can be accommodated. This overcomes the problems of ejecting UV filters encountered with SLNs. However, NLCs, due to the presence of solid lipids, exhibit the same type of spreading defect and heat sensitivity as SLNs.

Although their greater capacity to encapsulate UV filters and their better stability make NLCs more suitable than SLNs in the field of formulating sunscreen or anti-photoaging products, their stability during storage and after spreading on the skin is still not entirely satisfactory.

Another approach is represented by nanocapsules comprising an oily core and UV filters surrounded by a polymeric envelope (also called shell); this approach is described in numerous documents, such as French Patent Publication No. FR 3 009 682 (Polaar SAS and Université Claude Bernard).

These polymeric shells can be of different natures.

For example, document WIPO Patent Application Publication No. WO 2009/091 726 (Dow Global Technologies) describes the encapsulation of hydrophobic sunscreen molecules in polyurea shells obtained by interfacial polymerization of an isocyanate with an amine. The optimal shell thickness depends on the diameter of the microcapsules: for a diameter up to 4 μm, the shell must have a thickness greater than 10 nm, while for a size greater than 10 μm the shell must have a thickness of at least 100 nm.

WIPO Patent Application Publication No. WO 2013/059166 (Dow Global Technologies) describes a microencapsulated sunscreen in which the UV filter is encapsulated in polymeric microcapsules resulting from the reaction between an isocyanate prepolymer and water to form a polyurea shell. These microcapsules can be smaller than 1 μm, and regarding their thickness, the teaching of this document is similar to that of the previous document.

WIPO Patent Application Publication No. WO 2014/132261 (Tagra Biotechnologies) describes microcapsules formed from a polyacrylate, a polymethacrylate, a cellulose ether, a cellulose ester or a mixture of these polymers, containing UV filters. WIPO Patent Application Publication No. WO 2012/004461 (Biosynthis) describes microcapsules formed by polymerization of methyltrimethoxysilane or methyltriethoxysilane, containing UV filters.

There is a need for UV filter encapsulation systems and sunscreen product formulations with improved sun protection performance, while reconciling several other parameters, such as: good distribution of sunscreens between UVA and UVB with a minimum UVA/UVB intensity ratio of 33% for UVA protection on the skin and a critical wavelength greater than 370 nm, prevention of transcutaneous passage of UV filters, improved aesthetic properties such as the reduction of white marks left on the skin, particularly by mineral UV filters, after application.

It is also desirable to have sunscreen formulations that use as little as possible of substances called sunscreens to achieve a given sun protection factor.

### SUMMARY

The present invention aims to provide cosmetic formulations comprising micro-encapsulated UV filters, said formulations offering protection in the UVA and UVB spectra which meet all these requirements.

The present invention proposes to meet these aforementioned requirements, thanks to a selection of core-shell type microcapsules, comprising a shell encapsulating at least one active substance, which are very small in size, i.e. whose D_{S50} value does not exceed 1 μm, is preferably less than 1.0 μm and preferably remains less than 0.80 μm, but which only penetrates as far as the stratum corneum (i.e. the upper layer of the epidermis). These microcapsules are also water-resistant and mechanically strong against crushing and friction, while not giving a sticky appearance to the cosmetic compositions including said microcapsules. This strength is achieved through the use of a crosslinked polymer for the shell, and thanks to a sufficient thickness of the shell which represents at least 20% of the total mass of the microcapsule.

The microcapsules of the present invention are capable of being obtained by interfacial polymerization (and in particular by interfacial polymerization of a precursor of a polyurea-type polymer compound), so as to enclose an active substance such as a sunscreen.

The invention relates to cosmetic and/or dermo-cosmetic and/or pharmaceutical compositions comprising the microcapsules according to the invention, and in particular sun protection compositions or compositions claiming protection against photo-aging of the skin or skin appendages, preferably water-resistant. These formulations may be emulsions; in particular, they may be “oil-in-water emulsion” or “water-in-oil emulsion” type formulations.

In the context of the present invention, the compositions comprising the microcapsules according to the invention, preferably sun protection cosmetics or cosmetics claiming protection against photo-aging of the skin or skin appendages, can be formulated in particular in the form of liquid or pasty emulsions: ointments, creams, milks, mousses, or in the form of solid emulsions: sticks, bars, breads, or in non-emulsified form: oils, suspensions, gels, lotions, loose or compact powders and more generally, with other active ingredients, in the form of shower gel, shampoo, etc.

A first object of the invention is a core-shell type microcapsule, comprising a core containing at least one active ingredient, and comprising a shell, said shell constituting a wall around said core and representing a mass fraction of at least 20%, preferably at least 25%, more preferably at least 28%, even more preferably at least 30%, and even more preferably at least 32% (or even at least 35%), of the total mass of the microcapsule, said microcapsule being characterized in that the shell comprises at least one crosslinked polymer. Said microcapsule is preferably prepared by interfacial polymerization.

Said crosslinked polymer is advantageously chosen from polyurethane and/or polyurea.

The shell may further comprise an anionic surfactant, preferably alkyl ether sulfate, and/or at least one non-ionic surfactant, such as esters or ethers of polyethylene glycol, polyglycerol esters, esters of sorbitol derivatives such as, for example, sorbitan stearate, sucrose esters or a polysorbate, for example polysorbate 20, polysorbate 40 or polysorbate 60. Polysorbate 20 is the preferred nonionic surfactant.

The said core is advantageously liquid.

A second object is the use of the core-shell microcapsules according to the invention for the preparation of a sun protection formulation or a formulation providing protection against photo-aging of the skin and skin appendages. This formulation is preferably usable in the form of liquid or pasty emulsions (for example ointments, creams, milks, mousses), or in the form of solid emulsions (for example sticks, bars, breads), or in non-emulsified form (for example oils, suspensions, gels, lotions, loose or compact powders).

A third object of the invention is a sun protection or anti-photoaging formulation comprising a plurality of core-shell type microcapsules according to the invention. This formulation is presented in particular in the form of liquid or pasty emulsions (for example ointments, creams, milks, mousses), or in the form of solid emulsions (for example sticks, bars, breads), or in non-emulsified form (for example oils, suspensions, gels, lotions, loose or compact powders), comprising a plurality of microcapsules according to the invention. Advantageously, said sun protection or anti-photoaging formulation comprises at least 5% by mass, preferably at least 10% by mass, more preferably at least 20% by mass, even more preferably at least 30% by mass, and even more preferably at least 35% by mass of core-shell microcapsules relative to the total weight of said formulation.

Said sun protection or anti-photo-aging formulation according to the invention may have a sun protection factor SPF of at least 10, preferably at least 15, more preferably at least 30, and even more preferably at least 40, and optimally at least 50.

Yet another object of the invention is a method of manufacturing by interfacial polymerization core-shell microcapsules comprising a core containing at least one active ingredient and a shell, said shell constituting a wall around said core, representing a mass fraction of at least 20%, preferably at least 25%, even more preferably at least 30% and even more preferably at least 35% of the total mass of the microcapsule, and comprising at least one crosslinked polymer obtained from at least two precursor compounds A and B, said method comprising the steps consisting of:
(a) mixing said at least one active ingredient, a solvent and at least one compound A comprising more than two functional groups A′, to obtain a lipophilic phase;
(b) introducing, with stirring, the lipophilic phase obtained in step (a) into a continuous aqueous phase, which preferably comprises NaCl, to form an emulsion; and
(c) maintaining, with stirring, said emulsion at a temperature of between approximately 35° C. and approximately 90° C., preferably between approximately 40° C. and approximately 70° C., and even more preferably between approximately 55° C. and approximately 65° C., and introducing, with stirring, an aqueous solution comprising at least one compound B comprising more than two functional groups B′ to the emulsion obtained at the end of step (b) so as to react the functional groups A′ of said compound A comprising more than two functional groups A′ with the functional groups B′ of compound B comprising more than two functional groups B′, to form the crosslinked polymer shell constituting a wall around said core and thus to obtain core-shell microcapsules.

In an advantageous embodiment, the stirring during step (b) is carried out with a greater tangential speed than the stirring during the introduction of said aqueous solution comprising at least one compound B in step (c), and preferably with a tangential speed of at least 10 m/s, and even more preferably between 10 m/s and 30 m/s.

In an advantageous embodiment of this method, the continuous aqueous phase comprises at least one anionic surfactant and/or one non-ionic surfactant, said anionic surfactant preferably being chosen from alkyl ether sulfates, and even more preferably chosen from magnesium lauryl ether sulfate and sodium lauryl ether sulfate.

In another embodiment of this method, which may be combined with the preceding and with the other embodiments described herein, the NaCl content in the emulsion obtained in step (b) is between 0.10% and 1% by mass, and preferably between 0.15% and 0.90% by mass.

In yet another embodiment, which may be combined with the preceding and with the other embodiments described herein, the crosslinked polymer is selected from polyurea or polyurethane.

Yet another object of the invention is a method of manufacturing by interfacial polymerization core-shell microcapsules comprising a core containing at least one sunscreen and a shell constituting a wall around said core, said shell comprising a crosslinked polymer and at least one alkyl ether sulfate, the method comprising the steps of:
(a) mixing said at least one sunscreen, a solvent and a compound A comprising more than two functional groups A′, precursor of the crosslinked polymer shell constituting a wall around said core, to obtain a lipophilic phase;
(b) introducing, with stirring, the lipophilic phase obtained in step (a) into an aqueous continuous phase comprising NaCl and at least one alkyl ether sulfate, to form an emulsion; and
(c) maintaining, with stirring, said emulsion at a temperature of between approximately 35° C. and approximately 90° C., preferably between approximately 40° C. and approximately 70° C., and even more preferably between approximately 55° C. and approximately 65° C., and introducing, with stirring, an aqueous solution comprising a compound B comprising more than two functional groups B′ to the emulsion obtained at the end of step (b) so as to react the functional groups A′ of the compound A comprising more than two functional groups A′ with the functional groups B′ of the compound B comprising more than two functional groups B′, to form the crosslinked polymer shell constituting a wall around said core, and thus to obtain core-shell microcapsules.

### DRAWINGS

FIGS. 1 to 12 relate to the invention, of which they illustrate different aspects.

FIG. 1 refers to Example 1 and shows the particle size distribution of a batch of microcapsules with the reference 108, expressed in number (curve (a)), surface area (curve (b)) and volume (curve c)).

FIG. 2 relates to Example 1 and shows the particle size distribution of a batch of microcapsules with the reference 110, expressed in number (curve (a)), in surface area (curve (b)) and in volume (curve c)).

FIG. 3 refers to Example 1 and compares the surface particle size distribution between sample 108 (curve (a)) and sample 110 (curve (b)).

FIG. 4 relates to Example 3 and shows the SPF (Solar Protection Factor) as a function of the diameter of the microcapsules (expressed as DS50) according to a surface-weighted distribution according to the invention, all other things being equal.

FIG. 5 refers to Example 4 and shows the volume-weighted DV50 particle size distribution of microcapsules in a slurry.

FIG. 6 refers to Example 4 and shows the surface-weighted DS50 particle size distribution of microcapsules in the same slurry that generated FIG. 5.

FIG. 7 relates to Example 4 and shows the zeta potential which expresses the electric charge state of the surface of a particle within a colloid.

FIG. 8 relates to Example 5 and shows the photostability as a function of the number of irradiations (in 30-minute intervals) of a composition according to the invention comprising approximately 25% UV filter (curves c and d) and of a non-microencapsulated composition according to the state of the art (curves a and b) comprising the same concentration of the same active ingredient.

FIG. 9 relates to Example 5 and shows the photostability as a function of the number of irradiations (in 30-minute intervals) of a composition according to the invention comprising approximately 35% UV filter (curves c and d) and of a non-microencapsulated composition according to the state of the art (curves a and b) comprising the same concentration of the same active ingredient.

FIG. 10 relates to Example 5 and shows the SPF factor as a function of the UV filter concentration for a microencapsulated composition according to the invention (curve a) and a non-microencapsulated composition (curve b) comprising the same concentration of the same active ingredient.

FIG. 11 refers to Example 6 and shows fluorescence micrographs (magnification factor: 20) of human skin explant samples prepared in a cryomicrotome. On the left a sample taken from untreated skin, in the middle a sample taken from skin treated with a non-microencapsulated formulation, on the right a sample taken from skin treated with a microencapsulated formulation according to the invention.

FIG. 12 refers to FIG. 11 and shows the total intensity of the images, for a magnification factor of 20 (right) and a magnification factor of 10 (left).

### DESCRIPTION

Unless otherwise stated, all percentage values refer to mass. Unless otherwise stated, all D50 values are D_{S50} values: the median or D_{S50} size is the size for which the cumulative function is equal to 50%; it is surface area-weighted. The choice of surface area weighting is most relevant when studying the activity of a certain dispersed component, as in the present context.

The microcapsules according to the invention have a liquid core, which represents the active substance to be encapsulated, and a shell made of a crosslinked polymeric material. This crosslinked polymer is chosen from polyurea and/or polyurethane. Said active substance comprises at least one active substance selected from UV filters.

We first describe the fabrication of microcapsules.

According to the invention, these microcapsules are prepared by an interfacial polymerization process.

In a first step (hereinafter also called “step (a)), the active ingredient to be encapsulated, intended to form the liquid core of the microcapsule, is mixed with a solvent if necessary and a compound A comprising more than two functional groups A′, which is one of the precursors of the crosslinked polymer shell constituting a wall around the core. This produces a lipophilic phase, i.e. a phase that is immiscible with water.

In a second step (also called here “step (b)), the lipophilic phase obtained at the end of the first step is introduced, with stirring, into a continuous aqueous phase, to form an emulsion.

This continuous aqueous phase preferably comprises NaCl.

In a third step (also called herein “step (c)”), said emulsion is maintained, with stirring, at a temperature of between approximately 35° C. and approximately 90° C., preferably between approximately 40° C. and approximately 70° C., and even more preferably between approximately 55° C. and approximately 65° C., and an aqueous solution comprising a compound B comprising more than two functional groups B′ is introduced, with stirring, into the emulsion obtained at the end of step (b) of compound B so as to react the functional groups A′ of compound A comprising more than two functional groups A′ with the functional groups B′ of compound B comprising more than two functional groups B′. The product of this reaction is a crosslinked polymer. It forms the shell or wall of the microcapsule. This produces core-shell microcapsules comprising a crosslinked polymer shell constituting a wall around said liquid core. Typically, the reaction medium is left stirring until all the monomers, and in particular the isocyanate, have been consumed.

The advancement of this process requires some control of the temperature of the reaction mixture. The temperature depends on the reactivity of the monomers or prepolymers used, and on the agitation.

In a very advantageous embodiment of the invention, the stirring during step (b) is carried out with a greater tangential speed than the stirring during step (c), and preferably with a tangential speed of at least 10 m/s, and preferably between 10 m/s and 30 m/s. It should be noted that generally speaking, the number of revolutions of a rotor per unit of time is not a parameter suitable for characterizing the shear experienced by the liquid phase which is agitated by this rotor. It is the tangential velocity of an extreme point of the rotor which best and simply describes the shear in an emulsion chamber.

According to the invention, step (b) requires strong agitation, ensuring strong shear. This agitation is obtained, in a known manner, by the use of a rotor, and it is characterized in a simple manner by the tangential speed of the rotor. This strong shear is advantageously obtained with a tangential speed of at least approximately 10 m/s, and which is advantageously between approximately 10 m/s and approximately 30 m/s. Below about 10 m/s the microcapsules obtained have too large a diameter, which does not allow the benefits of a small diameter to be taken advantage of. Above about 30 m/s, controlling the emulsion temperature becomes difficult, due to excessive local heating of the rotor-stator shears.

Preferably, the tangential velocity is between approximately 10 m/s and approximately 28 m/s, more preferably between approximately 15 m/s and approximately 25 m/s, and even more preferably between approximately 15 m/s and approximately 23 m/s.

The third step typically begins with a period during which the temperature of the mixture is regulated to a desired value, preferably while maintaining the reaction mixture under vigorous stirring, i.e. at least initially within the same limits as in the second step. The addition of the aqueous solution comprising at least one compound B in step (c) is carried out with stirring characterized by a lower tangential speed than the stirring during step (b).

Thus, a tangential velocity when adding compound B of between about 1 m/s and about 6 m/s is suitable; preferably it is between about 2 m/s and about 5 m/s. The duration of the third stage must be sufficient for the emulsion formed to have the desired fineness and homogeneity, knowing that during polymerization, the microcapsules which form have a size comparable to that of the droplets of the emulsion.

In order for the addition of said aqueous solution to take place within the target temperature range during step (c), it is typically necessary to regulate this temperature. In particular, it is possible to preheat during step (b) said dispersed phase and/or said aqueous continuous phase, and/or it is also possible to heat the emulsion at the start of step (b). Since shearing of the emulsion provides thermal energy, in certain cases it may be necessary to cool the emulsion. For temperature management during the polymerization reaction in step (b) the exothermic nature of this reaction must be taken into account.

It is advantageous for the emulsion at the end of step (b) to be transferred into a different reactor, which is, on the one hand, provided with stirring means different from those of the reactor in which step (b) took place, and which is, on the other hand, provided with specific heat exchange means.

This process can be carried out in different ways.

According to one embodiment, in the second step, said lipophilic phase is brought to a temperature of between approximately 35° C. and approximately 90° C., preferably between approximately 40° C. and approximately 70° C., and even more preferably between approximately 55° C. and approximately 65° C.; in the third step, said continuous aqueous phase is at a temperature of between approximately 35° C. and approximately 90° C., preferably between approximately 40° C. and approximately 70° C., and even more preferably between approximately 55° C. and approximately 65° C.

Advantageously, in the third step, the temperature of said aqueous solution is similar to the temperature of said emulsion. It is preferred that their temperature does not differ by more than 15° C., preferably not more than 10° C., and even more preferably not more than 5° C.

The core of the microcapsules is advantageously liquid. Said continuous aqueous phase may comprise at least one anionic surfactant and/or at least one non-ionic surfactant. An advantageous anionic surfactant can be selected from the group consisting of: sodium lauryl sulfate, sodium C14-C16 olefin sulfonate, sodium laureth sulfate, disodium laureth sulfosuccinate, sodium methyl cocoyl taurate, sodium cocoyl isethionate, sodium lauryl sarcosinate, sodium lauroyl glycinate, sodium lauroyl lactate, magnesium laureth sulfate, TEA lauryl sulfate, sodium lauroyl glutamate, sodium laureth carboxylate, sodium laureth phosphate, sodium laureth sulfoacetate, hydrogenated lecithin; knowing that these names are those of the INCI (International Nomenclature of Cosmetic Ingredients).

In particular, the anionic surfactant may comprise an alkyl ether sulfate. A particularly advantageous surfactant comprises disodium-2-sulfolaurate; one such product is known under the trademark Texapon™.

It is preferred to use a non-ionic surfactant, which may be a polyethylene glycol ester or ether, a polyglycerol ester, an ester of sorbitol derivatives such as sorbitan stearate, a sucrose ester or an alkyl ether sulfate or polysorbate, for example polysorbate 20 (polyoxyethylene sorbitan monolaurate), also known as Tween™20, polysorbate 40 or polysorbate 60. Advantageously, a non-ionic surfactant may be selected from the group consisting of: Poloxamer 407, poloxamer 188, oleth-10, oleth-20, laureth-23, laureth-4, PEG-40 hydrogenated castor oil, PEG-60 hydrogenated castor oil, polysorbate 20, polysorbate 40, poysorbate 60, polysorbate 80, sorbitan oleate, sorbitan stearate, sorbitan palmitate, sorbitan myristate, sorbitan laurate, gluceth-20, PEG-7 glyceryl cocoate, PEG-6 caprylic capric glycerides, PPG-1-PEG-9 lauryl glycol ether, hepthyl glucoside, polyglyceryl-10 laurate, glyceryl oleate, polyglyceryl-4-oleate, PEG/PPG 20/23 dimethicone, PEG/PPG-23/6 dimethicone, PEG-8 dimethycone, dimethicone PEG-10 phosphate, sucrose laurate, sucrose palmitate; knowing that these names are those of the INCI.

These surfactants are located after polymerization in the shell.

According to an advantageous embodiment, the continuous aqueous phase used in this third step comprises at least one alkyl ether sulfate. Magnesium lauryl ether sulfate and sodium lauryl ether sulfate are preferred for this purpose.

According to another advantageous aspect of the invention, the NaCl content in said continuous aqueous phase used in step (b) is chosen so that the emulsion obtained at the end of this step (b) has an NaCl content of between 0.10% and 1% by mass, and preferably between 0.15% and 0.90% by mass. The addition of NaCl allows for more concentrated suspensions without aggregates.

According to an essential feature of the invention, said shell comprises at least one crosslinked polymer. This produces microcapsules that exhibit good mechanical robustness.

It is therefore necessary to choose appropriate monomers or oligomers which make it possible to obtain a crosslinked polymer. The functional groups A′ of the compound A comprising more than two functional groups A′ are chosen from isocyanate groups, in particular from polyisocyanates, which are molecules containing two or more isocyanate groups, such as diisocyanates. Preferred are diisocyanates and triisocyanates, whose isocyanate groups can be linked to an aliphatic or aromatic skeleton. The aliphatic polyisocyanates may be selected from aliphatic polyisocyanates containing two, three or more isocyanate functions, or mixtures of these polyisocyanates. Preferably, the aliphatic polyisocyanate comprises one or more cycloalkyl skeletons.

For example, compound A may be selected from the group consisting of:
dicyclohexylmethane 4,4′-diisocyanate, hexamethylene 1,6-diisocyanate, isophorone diisocyanate, trimethyl-hexamethylene diisocyanate, trimer of hexamethylene 1,6-diisocyanate, isophorone trimer diisocyanate, 1,4-cyclohexane diisocyanate, 1,4-(dimethylisocyanato) cyclohexane, hexamethylene diisocyanate biuret, hexamethylene diisocyanate biuret (CAS No. 4035-89-6), trimethylene diisocyanate, propylene-1,2-diisocyanate, butylene-1,2-diisocyanate, tetramethylene diisocyanate, pentamethylene diisocyanate, hexamethylene diisocyanate, 4-(isocyanatomethyl)-1,8-octyl diisocyanate;
mixtures between aliphatic diisocyanates and aliphatic triisocyanates,
aromatic polyisocyanates such as 2,4-toluene diisocyanate, 2,6-toluene diisocyanate, naphthalene diisocyanate, diphenylmethane diisocyanate, triphenylmethane-p,p′,p″-trityl triisocyanate,
aromatic isocyanates such as toluene diisocyanate, polymethylene polyphenylisocyanate, 2,4,4′-diphenyl ether triisocyanate, polymethylene polyphenylisocyanate, 2,4,4′-diphenyl ether triisocyanate, 3,3′-dimethyl-4,4′-diphenyl diisocyanate, 3,3-dimethoxy-4,4′-diphenyl diisocyanate, 1,5-naphthalene diisocyanate, 4,4′,4″-triphenylmethane triisocyanate, isophoron diisocyanate.

The functional groups B′ of compound B comprising more than two functional groups B′ are chosen so that compound B is an amine (such as diamines or polyamines). For example, one or more of the following amines may be used: ethylene diamine, diethylene triamine, propylene diamine, tetraethylene pentaamine, pentamethylene hexamine, alpha omega diamine, propylene-1,3-diamine, tetramethylene diamine, pentamethylene diamine, 1,6-hexamethylene diamine, triethylene triamine, pentaethylene hexamine, 1,3-phenylene diamine, 2,4-toluylene diamine, 4,4′-diaminodiphenyl methane, 1,5-diamino naphthalene, 1,3,5-triaminobenzene, 2,4,6-triaminotoluene, 1,3,6-triamino naphthalene, 2,4,4′-triaminodiphenyl ether, 3,4,5-triamino-1,2,4-triazole, bis(hexamethylene triamide), 1,4,5,8-tetraamino anthraquinone.

A preferred amine is guanidine carbonate.

According to an advantageous embodiment, an amount of prepolymer in the oily phase which is greater than 25% by volume is used in the interfacial polymerization process.

According to an essential feature of the invention, said shell constituting a wall around said core represents a mass fraction of at least 20%. Advantageously this fraction is between approximately 20% and approximately 45%, and more preferably between approximately 25% and approximately 35%.

With some isocyanates leading to a relatively hard shell, the mass fraction of the wall around the core advantageously represents between about 28% and about 32% of the total mass of the microcapsule.

According to an advantageous aspect of the invention, the microcapsules have a size DS50 less than or equal to 0.80 μm. This makes it possible to benefit from an effect that the inventors discovered unexpectedly: for a sun protection or anti-photo-aging formulation comprising a given content of sun filter, the sun protection factor SPF increases when the size DS50 of the microcapsules containing this formulation is less than or equal to 0.80 μm.

This allows to either obtain a higher SPF factor with a given content of sunscreen formulation, or to obtain a given SPF factor with a lower quantity of sunscreen formulation.

A DS50 size less than or equal to 0.75 μm is preferred, even more preferably less than or equal to 0.70 μm, and even more preferably less than or equal to 0.65 μm, or even less than or equal to 0.60 μm, because below a DS50 size of approximately 0.8 m, the SFP factor increases when the size of the microcapsules decreases.

At the end of the third step, a suspension is obtained comprising a dispersed phase (called “slurry” by those skilled in the art) comprising the microcapsules, and a liquid phase comprising solvents, surfactant, unreacted reagents, as well as various secondary reaction products. In a typical embodiment, the slurry comprises about 20% by mass of microcapsules. The microcapsules are washed and concentrated according to methods known to those skilled in the art. After removal of the continuous phase, a suspension can be obtained having more than 50% by mass of microcapsules, preferably between approximately 50% and approximately 70%, and even more preferably between approximately 60% and approximately 70%, the remainder being water (as well as various residues). This suspension can be used directly for the preparation of sunscreen or anti-photoaging formulations incorporating the microcapsules according to the invention; this can be done using known methods, as will be illustrated in the examples. It is also possible to continue drying to obtain a microcapsule powder.

In order to avoid softening of the microcapsules and their rupture, the microcapsules must be introduced with moderate stirring, and preferably at a temperature not exceeding that at which the emulsion was prepared (third step of the process described above), and even more preferably at a temperature below 60° C.

Generally, basic ingredients, active ingredients as well as cosmetic excipients and additives of known types can be used. The nature and quantities of sunscreens used must comply with current legislation on compositions containing sunscreens depending on the country of marketing. For example, when marketing a sunscreen formulation in the USA, an avobenzone concentration of 3% must not be exceeded.

The microcapsules according to the invention can contain all types of active substances (also called “active ingredients”). We give here, for information purposes, a list of active ingredients capable of acting as sunscreens, which are preferred for carrying out the present invention. These active ingredients are identified here by their INCI name (International Nomenclature of Cosmetic Ingredients) and by their CAS number:

Glyceryl PABA (CAS No. 136-44-7), Menthyl antranilate (CAS No. 134-09-8), Diethylamino hydroxybenzoyl hexylbenzoate (CAS No. 302776-68-7), Polysilicone-15 (CAS No. 207574-74-1), Bis-ethylhexyloxyphenol methoxyphenyl triazine (CAS No. 187393-00-9-6), Ethylhexyl dimethyl PABA (CAS No. 21245-02-3), Ethylhexyl salicylate (CAS No. 11860-5), 3-Benzylidene camphor (CAS No. 15087-24-8), Diethylhexyl butamimido triazone (CAS No. 154702-15-5), 4-Methylbenzylidene camphor (CAS No. 38102-62-4/36861-47-9), PABA (CAS No. 150-13-0), Homosalate (CAS No. 118-56-9), Benzophenone-3 (CAS No. 131-57-7), Butyl methoxydibenzoylmethane (CAS No. 70356-09-1), Octocrylene (CAS No. 6197-30-4), Ethylhexyl methoxycinnamate (CAS No. 5466-77-3), Isoamyl p-methoxycinnamate (CAS No. 71617-10-2), Ethylhexyl triazone (CAS No. 88122-99-0), Drometrizole Trisiloxane (CAS No. 155633-54-8), Phenylene bis-diphenyltriazine (CAS No. 55514-22-2).

Stabilizing agents can be added, which can be of known types.

Formulations containing Butyl methoxydibenzoylmethane gain photostability if at least one of the following ingredients is added to the formulation of the microencapsulated phase: butyloctyl salicylate, C₁₂₋₁₅ alkyl benzoate, diethylhexyl 2,6-naphthalate, polyester-8, ethylhexyl methoxycrylene, octocrylene, diethylhexyl syringylidene malonate, tocopherol, triethylcitrate.

This results in formulations for sun protection or against photo-aging which have numerous advantages. These formulations are highly resistant to spreading on the skin, as the microcapsules withstand high shear forces in an aqueous environment when applied to the skin. These formulations minimize the penetration of the sunscreen into the skin, as the microcapsules that contain it are mechanically very stable. These formulations have, for a given concentration of sunscreen, a higher sun protection factor. These formulations can be made with an SPF factor that varies within fairly wide limits. It is preferred that the SPF be at least 10, preferably at least 15, more preferably at least 30, and even more preferably at least 40. Formulations with an SPF greater than 50 can be made.

### EXAMPLES

### Example 1: Preparation of Microcapsules

Here we describe nine examples of implementation of the method according to the invention.

### Example 1-1

Monomer A was an aliphatic polyisocyanate. Monomer B was guanidine carbonate. The active ingredient to be encapsulated was a mixture of butyl methoxydibenzoylmethane (15%), octocrylene (15%) and homosalate (70%).

A lipophilic phase was prepared which contained 69.7% of the active ingredient mixture (sunscreens) to be encapsulated with 30% of monomer A and 0.3% of a non-ionic surfactant SPAN™ 60. A continuous aqueous phase was prepared with 6500 mL of demineralized water, 1% of the surfactant Texapon™ and 0.3% of NaCl. A 500 mL solution was prepared with a stoichiometric amount of monomer B.

The lipophilic phase and the continuous aqueous phase were heated to 60° C. At this temperature, the lipophilic phase to be dispersed was added to the hot continuous phase in a circuit containing a rotor stator with very high shear, for 6 min, at a rate of 20% of lipophilic phase in the continuous aqueous phase containing the surfactant. The dispersion of the circuit was then emptied into a thermostatically controlled tank at 60° C. with constant gentle stirring. The solution of monomer B was added slowly, and the temperature was maintained at 60° C. for at least 6 hours.

The D_{X50(surface)} value of the microcapsules was 680 nm.

### Example 1-2

Monomer A was a mixture of an aliphatic polyisocyanate (80%) and a polyphenyl-polymethylene polyisocyanate (20%). Monomer B was guanidine carbonate. The active ingredient to be encapsulated was a mixture of butyl methoxydibenzoylmethane (13%), ethylene salicylate (22%), homosalate (55%) and propylene carbonate (10%).

A lipophilic phase was prepared which contained 69.7% of the active ingredient mixture (sunscreens) to be encapsulated with 30% of monomers A and 0.3% of a non-ionic surfactant SPAN™ 60. An aqueous continuous phase was prepared with 6500 mL of deionized water, 0.5% of the surfactant Texapon™, 1% of the surfactant Tween™80 and 0.1% NaCl. A 500 mL solution was prepared with a stoichiometric amount of monomer B.

The other steps of the process were carried out as described in relation to Example 1-1: The lipophilic phase and the aqueous continuous phase were heated to 60° C. At this temperature, the lipophilic phase to be dispersed was added to the hot continuous phase in a circuit containing a rotor stator with very high shear, for 6 min, at a rate of 20% of lipophilic phase in the continuous aqueous phase containing the surfactant. The dispersion of the circuit was then emptied into a tank thermostatically controlled at 60° C. with constant gentle agitation. Monomer B solution was added slowly, and the temperature was maintained at 60° C. for at least 6 hours.

The D_{X50(surface)} value of the microcapsules was 840 nm.

### Example 1-3

Monomer A was a mixture of an aliphatic polyisocyanate (60%) and a polyphenyl-polymethylene polyisocyanate (40%). Monomer B was guanidine carbonate. The active ingredient to be encapsulated was a mixture of butyl methoxydibenzoylmethane (15%), ethylhexyl dimethyl PABA (15%) and homosalate (70%).

A lipophilic phase was prepared which contained 69.8% of the active ingredient mixture (sunscreens) to be encapsulated with 30% of monomers A and 0.2% of a non-ionic surfactant SPAN™ 60. An aqueous continuous phase was prepared with 6500 mL of demineralized water, 1% of the surfactant Tween™20 and 0.1% of NaCl. A 500 mL solution was prepared with a stoichiometric amount of monomer B.

The other steps of the process were carried out as described in connection with Example 1-1.

The D_{X50(surface)} value of the microcapsules was 720 nm.

### Example 1-4

Monomer A was a mixture of an aliphatic polyisocyanate (90%) and a polyphenyl-polymethylene polyisocyanate (10%). Monomer B was a mixture of guanidine carbonate (90%) and glycerol (10%). The active ingredient to be encapsulated was a mixture of butyl methoxydibenzoylmethane (13%), ethylhexyl salicylate (18%), homosalate (65%) and tocopheryl acetate (4%).

A lipophilic phase was prepared which contained 69.7% of the active ingredient mixture (sunscreens) to be encapsulated with 30% of monomers A and 0.3% of a non-ionic surfactant SPAN™ 60. A continuous aqueous phase was prepared with 6500 mL of demineralized water, 1% of the surfactant Texapon™ and 0.3% of NaCl. A 500 mL solution was prepared with a stoichiometric amount of monomers B.

The other steps of the process were carried out as described in connection with Example 1-1.

The D_{X50(surface)} value of the microcapsules was 750 nm.

### Example 1-5

Example 1-1 was repeated with the following changes: Monomer A was an aliphatic polyisocyanate. Monomer B was guanidine carbonate. The active ingredient to be encapsulated was a mixture of butyl methoxydibenzoylmethane (15%), ethylhexyl triazone (8%), dibutyl adipate (69%), tocopheryl acetate (8%).

The D_{X50(surface)} value of the microcapsules was 450 nm.

### Example 1-6

Example 1-1 was repeated with the following changes: Monomer A was a mixture of an aliphatic polyisocyanate (90%) and a polyphenyl-polymethylene-polyisocyanate (10%).

Monomer B was a mixture of guanidine carbonate (90%) and lysine (10%). The active ingredient to be encapsulated was a mixture of butyl methoxydibenzoylmethane (15%), ethylhexyl triazone (7%), dibutyl adipate (55%), ethylhexyl methoxycinnamate (15%), polyester-8 (8%).

The D_{X50(surface)} value of the microcapsules was 560 nm.

### Example 1-7

Monomer A was a mixture of an aliphatic polyisocyanate (50%) and a polyphenyl-polymethylene polyisocyanate (50%). Monomer B was guanidine carbonate. The active ingredient to be encapsulated was a mixture of butyl methoxydibenzoylmethane (13%), ethylhexyl triazone (7%), dibutyl adipate (55%), ethylhexyl methoxycinnamate (15%) and triethyl citrate (10%).

A lipophilic phase was prepared which contained 69.7% of the active ingredient mixture (sunscreens) to be encapsulated with 30% of monomers A, 1% of the surfactant SPAN™ and 1% of the surfactant Tween™20. A continuous aqueous phase was prepared with 6500 mL of demineralized water and 0.1% NaCl. A 500 mL solution was prepared with a stoichiometric amount of monomers B.

The other steps of the process were carried out as described in connection with Example 1-1.

The D_{X50(surface)} value of the microcapsules was 510 nm.

### Example 1-8

Monomer A was a mixture of an aliphatic polyisocyanate (64%) and a polyphenyl-polymethylene polyisocyanate. Monomer B was guanidine carbonate. The active ingredient to be encapsulated was a mixture of diethylamino hydroxybenzoyl hexylbenzoate (13%), ethylhexyl triazone (7%), bis-ethylhexyl-oxyphenol methoxyphenyl triazine (20%) and dibutyl adipate (60%).

A lipophilic phase was prepared which contained 69.7% of the active ingredient mixture (sunscreens) to be encapsulated with 30% of monomers A and 0.3% of the surfactant SPAN™60. A continuous aqueous phase was prepared with 6500 mL of demineralized water with 0.1% of the surfactant Texapon™, 1% of the surfactant Tween™80 and 0.3% NaCl. A 500 mL solution was prepared with a stoichiometric amount of monomer B.

The other steps of the process were carried out as described in connection with Example 1-1.

The D_{X50(surface)} value of the microcapsules was 670 nm.

### Example 1-9

Monomer A was a mixture of an aliphatic polyisocyanate (85%) and a polyphenyl-polymethylene polyisocyanate (15%). Monomer B was guanidine carbonate. The active ingredient to be encapsulated was a mixture of benzophenone-3 (12%), ethylhexyl triazone (8%), ethyl hexyl dimethyl PABA (20%), 4-methylbenzylidene camphor (20%) and isopropyl palmitate (40%).

A lipophilic phase was prepared which contained 69.7% of the active ingredient mixture (sunscreens) to be encapsulated with 30% of monomers A and 0.1% of the surfactant SPAN™60. A continuous aqueous phase was prepared with 6500 mL of demineralized water with 1% of the surfactant Texapon™ and 0.5% NaCl. A 500 mL solution was prepared with a stoichiometric amount of monomer B.

The other steps of the process were carried out as described in connection with Example 1-1.

### Example 2: Granulometry of Microcapsules with References 110 and 108

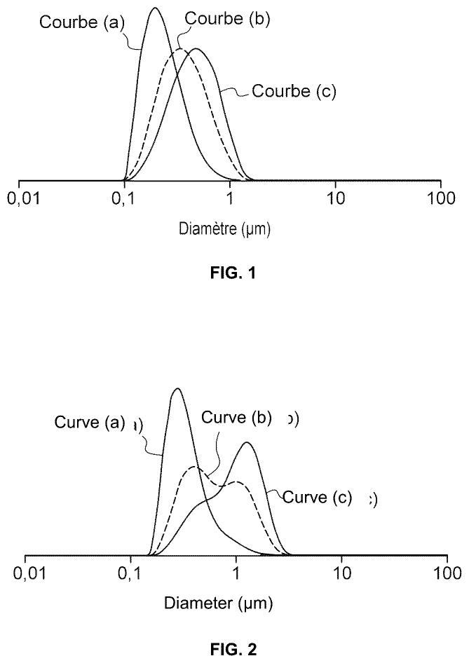

The water suspension and the surfactant containing the microcapsules according to the invention were diluted with demineralized water and analyzed with a Mastersizer™ 3000 Granulometer (Malvern). The results for the microcapsules with reference 108 are reported in FIG. 1, with the different representations provided by the instrument (Volume; Surface; Number). All distributions are monomodal. This indicates that the dispersion of microcapsules is rather homogeneous, although the difference between “number” and “volume” suggests a significant degree of poly-dispersion in microcapsule size. In all cases, the average size of the microcapsules is between 200 nm and 800 nm.

The results for the sample with reference 110 are reported in FIG. 2. In this case the distributions have a quasi-bimodal tendency (shoulder at 500 nm in the distribution in volume; distribution superposition of two Gaussians in the superficial area distribution), which suggests a greater poly-dispersion in size compared to sample 110, with the distinction of two families of microcapsules with clearly different sizes. This is evident from the surface distribution, with the two peaks coinciding with the median of the number (smaller size) and volume (larger size) distributions.

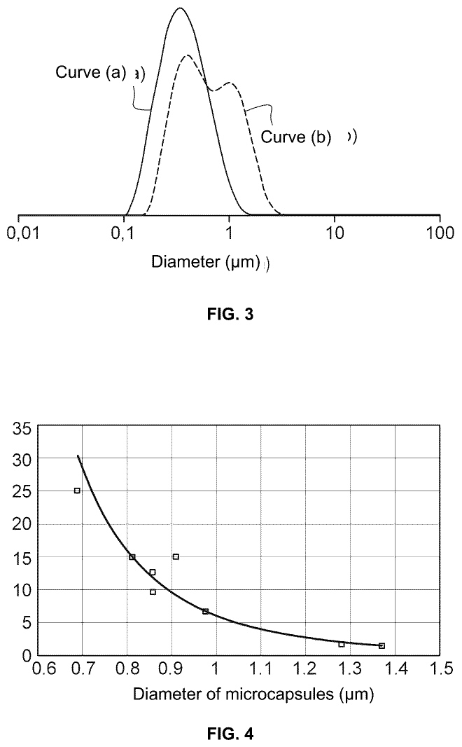

To better see the differences in the finely dispersed fraction between samples 110 and 108, the surface distributions were compared and the results are reported in FIG. 3. It can be clearly seen that the average surface area of the reference 108 microcapsules is significantly smaller compared to the reference 110 microcapsules, with a more homogeneous distribution as well.

The laser granulometry results on samples 110 and 108 show that these two types of microcapsules have a very fine size dispersion (above the micron). The microcapsules of reference 108 have a more uniform and finer size dispersion compared to the reference 110, which could give them greater effectiveness in UV protection when used in a cosmetic formulation.

These results also show that laser granulometry can be used to determine the average size of microcapsules in colloidal dispersion. It is a fast and inexpensive method, compared to electron microscopy.

A laser particle size analyzer is capable of measuring the size distribution of particles in a colloidal dispersion, using laser diffraction. The size of a particle can be described in several ways: by its maximum length, its minimum length, its volume, its surface area. Thus, the size distribution of a colloidal dispersion can also be represented in different modes, depending on the analysis technique used:
1) Number (D[1,0]): weighted to the smallest sizes (used rather in microscopy) (FIG. 1)
2) Volume (D[4,3]): weighted to the largest sizes (used rather in laser diffraction) (FIG. 1)
3) Surface area (D[3,2]): median between number and volume.

Volume representation is often used to observe differences between two colloidal dispersions, when focusing on the coarse fraction of the dispersion. The surface representation, on the other hand, is rather used when we want to know the differences in the finely dispersed fraction, useful especially when in applications where the surface area of the dispersed particles is important (drugs; cosmetics; paints).

Typically, in a laser granulometer the sample passes continuously under suction through the laser analysis chamber. The laser light scattered by the sample is intercepted by detectors located at different angles around the sample. This type of conformation allows to discriminate between different particle size families in a colloidal dispersion, because smaller particles scatter laser light at larger angles compared to larger particles. To avoid saturation of the detectors, the sample must have an appropriate degree of dilution.

### Example 3: Relationship Between Particle Size and SPF Factor of Microcapsules with Reference 110

The analyzed samples are slurries of microcapsules with reference 110 containing 20% by mass of a mixture of UV filters compatible with the requirements of the US FDA. The microcapsules are synthesized from different emulsions made by a rotor stator with different shear rates. These emulsions are then transformed by an interfacial polymerization process into microcapsules. Thus, different slurries of microcapsules were obtained which are characterized by a measurement of granulometry, which can be weighted either in number, in surface area, in volume or in mass.

Diameter size measurements are made with a Mastersizer™ 3000 granulometer (Malvern).

Before each measurement, the starting sample is diluted with demineralized water until an optimal level of saturation of the optical detectors is reached. Three measurement iterations are performed per sample reference. The obtained granulometry can be processed in three different ways: by volume, by number and by surface.

For this study, the results of the D_{S50} of the surface-weighted granulometry are retained.

The median or D_{S50} size is the size for which the cumulative function is equal to 50%; it is surface area-weighted. The choice of surface area weighting is most relevant when studying the activity of a certain dispersed component (paint; catalysis; pharma).

SPF measurements were carried out on these same samples, using the in vitro method on PMMA plates in order to relate D_{S50} with SPF.

The results are reported in FIG. 4 and Table 1.

TABLE 1

Diameter of microcapsules of Ds₅₀,
surface-area weighted [μm]

| | 1.37 | 1.28 | 0.98 | 0.86 | 0.81 | 0.69 |
|---|---|---|---|---|---|---|
| SPF | 1.5 | 1.8 | 6.7 | 12.6 | 15.0 | 25.1 |

FIG. 4 represents the curve of the measured SPFs as a function of the median diameter (D_{S50}) of the microcapsule slurries.

The results clearly show that when the D_{S50} of the microcapsules decreases the SPF of the suspension increases. This effect, as shown by the interpolation curve (red line) is exponential and gives good SPFs (relative to the amount of encapsulated filter) when the surface area-weighted median diameter (D_{S50}) is less than 800 nm.

These results suggest that when suspensions contain finely dispersed microcapsules, they are capable of absorbing UV rays more effectively for the same application surface and consequently of better protecting against the harmful effects of these same rays. This property is therefore essential to obtain a product that performs well in terms of effectiveness (SPF) for products containing microcapsules of sunscreens.

### Example 4: Characterization of the Zeta Potential of a Colloidal Suspension

In this study, the four samples analyzed are suspensions of microcapsules (MCs) of reference 110 with 20%. Each sample was made with a different amount of NaCl: 0% (110A); 0.35% (110B), 0.7% (110C) and 1.4% (110D).

For Zeta Potential measurements, suspensions containing type 110 microcapsules were pre-diluted with demineralized water by a factor of 1000, in order to obtain a clear and transparent dispersion, suitable for analysis. Three iterations of measurements are carried out for each sample with a Malvern Zetasizer Nano (Malvern Instruments).

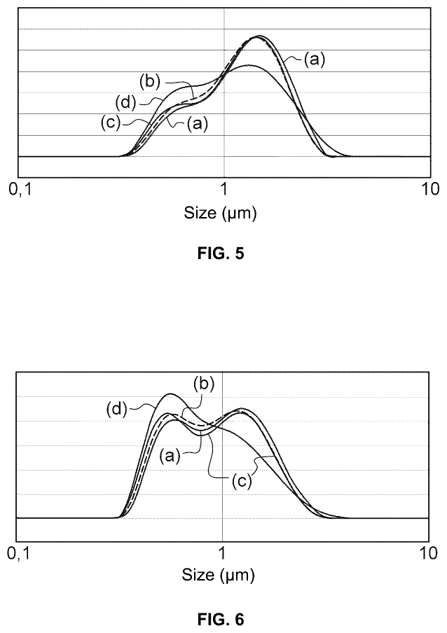

Diameter size measurements are carried out with a Mastersizer 3000 granulometer (Malvern). Before each measurement, the starting sample is diluted with demineralized water until an optimal level of saturation of the optical detectors is reached. The obtained granulometry can be weighted according to three different modes: by volume, by number and by surface. For this study, the results of the D_{S50} parameter of the surface area-weighted granulometry (see FIG. 6) and the Dv50 parameter of the volume-weighted granulometry (see FIG. 5) are retained. In FIGS. 5, 6 and 7, curve (a) corresponds to sample 110A, curve (b) to sample 110B, curve (c) to sample 110C and curve (d) to sample 110D.

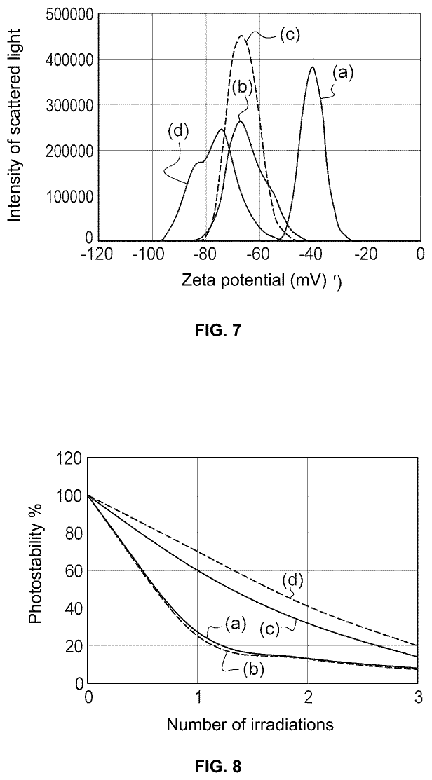

The Zeta Potential results in FIG. 7 clearly show a clear increase in the charge on the surface of the microcapsules which goes from −40 mV to −66 mV with the presence of salt during synthesis. This increase is however weakly dependent on the NaCl concentration, i.e. the charge varies very little as a function of salt concentration (0.35%; −67 mV; sample 110B) and the largest (1.4%; −74 mV; sample 110D).

The particle size results presented in FIGS. 5 and 6 clearly show that the increase in surface charge is due to the presence of salt; however, the size dispersion of the microcapsules is independent or almost independent of the presence of salt. Both the surface and volume distributions have a bimodal distribution which suggests the presence of two families of microcapsules with average sizes centered around 0.6 μm and 1.3 μm. The more finely dispersed family of microcapsules becomes the majority when the NaCl concentration reaches 1.4%.

This example shows that the presence of NaCl increases the negative zeta potential on the surface of the microcapsules independently of the concentration of the salt tested.

Therefore, the stability of microcapsule suspensions is directly impacted by the presence of salt during synthesis. The more intense surface charges stabilize microcapsule dispersions by promoting electrostatic repulsion and minimize aggregation effects. This effect can be achieved with a low salt concentration (0.35%). This explains why when synthesizing suspensions, more concentrated and aggregate-free suspensions can be obtained when salt is added.

### Example 5: Study of the Photostability of UV Filter Compositions According to the Invention

The results were obtained using specific in vitro methods for determining the SPF sun protection index, IIV-A protection and calculating the Critical Wavelength (abbreviated LOC) on Sunplate-type polymethyl methacrylate (PMMA) support.

Four measurements are carried out with a Kontron™ 933 spectrophotometer equipped with an integrating sphere in order to determine the sun protection factors. HelioTest® No. 1, HelioTest® No. 2, HelioTest® No. 3 and HelioTest® No. 5 methods were used to determine SPF, UVA and LOC values and to assess photostability.

The solar irradiation test was carried out with a Suntest Atlas CPS+ simulator at 550 W/m² in 30-minute intervals; the correspondence with the DEM unit (minimum erythema dose) is: 30 min=4 DEM, 60 min=8 DEM, 120 min=16 DEM. As known to those skilled in the art, the DEM unit expresses the smallest quantity of light capable of triggering sunburn at the location of exposure after 24 hours.

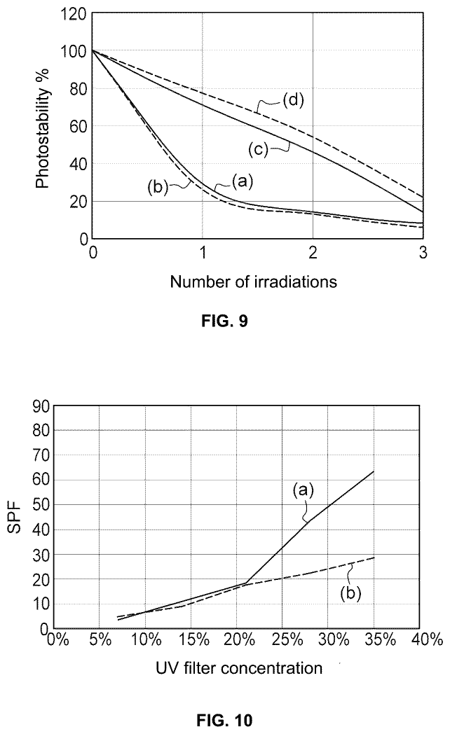

The results are collected in Table 2 below, as well as in FIGS. 8 and 9.

TABLE 2

| | | | | | LOC | Photo- |
|---|---|---|---|---|---|---|
| Sample | IR | SPF | SPF | | CM | stability |
| reference | [min] | measured | displayed | UVA | [nm] | [%] |
| 8S0F1-1021 | 0 | 23.8 | 20 | 18.2 | 378 | — |
| Oily phase | 30 | 6.0 | 6 | 4.1 | 380 | 25 |
| FDA Ref. | 60 | 3.1 | * | 1.8 | 378 | 13 |
| 5421_15L | 120 | 1.6 | * | 1.3 | 378 | 6 |
| (23%) | | | | | | |
| 22SOF1-1021 | 0 | 31.2 | 30 | 21.7 | 382 | — |
| Slurry μCaps | 30 | 21.7 | 20 | 13.0 | 380 | 69 |
| US Dry extract | 60 | 12.7 | 10 | 6.9 | 380 | 40 |
| 23% (23%) | 120 | 6.4 | 6 | 3.1 | 380 | 20 |
| 22SOF2-1021 | 0 | 28.4 | 25 | 16.7 | 378 | — |
| Oily phase | 30 | 7.5 | 6 | 4.9 | 380 | 26 |
| FDA Ref. | 60 | 3.8 | * | 2.4 | 378 | 13 |
| 5421_15L | 120 | 1.6 | * | 1.3 | 382 | 5 |
| (35%) | | | | | | |
| 8SOF2-1021 | 0 | 63.6 | 50+ | 56.7 | 382 | — |
| Slurry μCaps | 30 | 48.6 | 30 | 37.4 | 382 | 77 |
| US Dry extract | 60 | 34.6 | 30 | 22.0 | 381 | 55 |
| (35%) | 120 | 13.9 | 10 | 4.7 | 378 | 22 |

IR = Sun irradiation at 550 W/m² with Suntest Atlas CPS+ simulator.

FIG. 8 shows the evolution of the SPF factor as a function of the concentration of UV filter in the core of the microcapsule, and compares an encapsulated sample, with a sun filter content of 23% relative to the total mass of the microcapsule (curve (c): UV-A; curve (d): IIV-B), according to the invention, with the same non-encapsulated active substance (curve (a): UV-A; curve (b): UV-B) contained in a fatty phase according to the state of the art without microcapsules. The vertical axis represents the SPF value (100% being the initial value), the horizontal axis represents the number of 30-minute irradiation periods.

It is noted that the fatty phase is not stable under UV irradiation: this instability is total after two hours of irradiation at 550 W/m² (16 DEM). The suspension is stable during the first irradiations, and then develops an instability which becomes very significant after two hours of irradiation at 550 W/m² (16 DEM). The encapsulation of the UV filter improves the photostability of the formulation for both the SPF factor and UV-A protection.

FIG. 9 shows the same type of curve for a microcapsule representing a sunscreen content of 35%. The curves refer to a microencapsulated formulation according to the invention (curve (c): UV-A; curve (d): UV-B) and to a fatty formulation according to the state of the art without microcapsules (curve (a): UV-A; curve (b): UV-B).

The observations regarding the photostability of the fatty phase and the suspension are the same as for the previous figure. The fatty phase is not stable under UV irradiation: this instability is total after two hours of irradiation at 550 W/m (16 DEM). The suspension is stable during the first irradiations, and then develops an instability which becomes very significant after two hours of irradiation at 550 W/m² (16 DEM). The encapsulation of the UV filter improves the photostability of the formulation for both the SPF factor and UV-A protection.

FIG. 10 shows the evolution of the SPF factor measured for microencapsulated suspension compositions according to the invention (curve (a)) and for non-microencapsulated suspension compositions outside the invention (curve (b)), for compositions comprising different concentrations of the UV filter. It is noted that beyond a concentration of approximately 21% to 22%, the microencapsulated suspension shows a significantly higher protective activity than the non-encapsulated suspension, whereas for lower concentrations, no difference is seen.

### Example 6: Comparison of In Vitro Skin Absorption of Sunscreens in Encapsulated and Non-Encapsulated Form

A suspension of microcapsules concentrated at 33% in water and comprising 23% of encapsulated sunscreens, according to the invention, and a control of non-encapsulated sunscreens diluted at 23% in a product sold under the trademark Cétiol Ultimate (undecane/tridecane mixture) were used.

Human skin explants mounted on a Transwell insert in a 6-well plate were used. The treatment area was 1 cm². The receiving fluid was PBS (1 mL). The treatment volume was 10 μL/cm², the treatment duration was 24 hours at 37° C.

Three different treatments were used: a control treatment (without active ingredient), an encapsulated formulation according to the invention, and a non-encapsulated formulation outside the invention.

At the end of treatment, excess formulation was removed with cotton swabs. A biopsy was taken from the treated surface using a 100 mm diameter punch. The biopsy was cut in half using a scalpel, mounted in a cryomatrix and placed on the cryobar at −50° C. before being mounted on the cutting holder. Slices of 5 μm thickness were cut from each sample using the cryomicrotome.

The slices were observed under a fluorescence microscope using the DAPI filter, at a magnification of 10 and 20. For image acquisition the exposure time was 20 ms. Image analysis was performed using ImageJ software, which allows semi-quantitative analysis of fluorescence intensity.

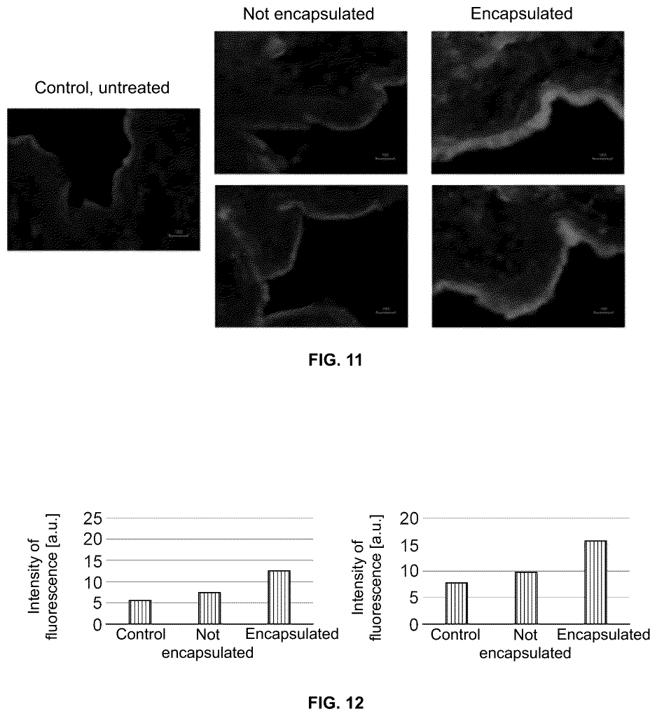

FIG. 11 shows fluorescence micrographs obtained for the three samples (two different areas for each of the treated samples) at a magnification of 20.

FIG. 12 shows the fluorescence signal, in arbitrary units, obtained by image analysis, for magnification 10 and magnification 20, of the three samples. More specifically, FIG. 12 shows the total intensity of the images in FIG. 11, for a magnification factor of 20 (right) and a magnification factor of 10 (left).

Regarding the untreated control sample, a diffuse fluorescent signal is observed which corresponds to the autofluorescence of the skin.

Concerning the sample treated with the non-encapsulated formulation, image analysis shows that the fluorescence intensity (diffuse signal) is slightly higher compared to the control sample.

Concerning the sample treated with the encapsulated formulation according to the invention, the signal is located at the level of the stratum corneum. The fluorescence intensity is much higher compared to the untreated control sample and to the sample treated with the unencapsulated formulation; this signal is therefore specific to the formulation according to the invention.

### Examples 7: Examples of Support Formulation for Sunscreens

### Example 7-1

The formulation was based on the following ingredients:

| Phase | Ingredient according to INCI nomenclature | % by mass |
|---|---|---|
| A | Suspension of microcapsules according to the | 66 |
| | invention, at 50% | |
| A | Citric acid | 0.5 |
| B | Xanthan gum | 0.1 |
| B | Hydroxypropyl guar | 0.1 |
| C | Aqua | 2.5 |
| C | Disodium EDTA | 0.05 |
| D | Sodium stearoyl glutamate | 1 |
| D | Cetyl alcohol | 1 |
| D | Cetearyl alcohol, Coco-glucoside, Aqua, Glucose | 2 |
| D | Dicaprylyl carbonate, Tocopherol | 8 |
| D | Squalane | 7 |
| D | Tricontanyl PVP | 2 |
| E | Undecane, Tridecane, Tocopherol | 8 |
| F | Tocopherol, Helianthus Annuus Seed Oil | 0.5 |
| G | Phenoxyethanol, Ethylhexylglycerin | 1 |
| H | Citric acid | 0.25 |

For this test a planetary stirrer was used, but no homogenizer. The components of phase A were cold mixed without using a homogenizer. Then the constituents of phase B were dispersed in phase A, still without using a homogenizer, then the ingredients of phase C, previously well dissolved, were added. Phase D was heated to 80° C. to mix its constituents well, then phase E (unheated) was added to phase D, before introducing this mixture of phases D and E, still without using a homogenizer, into the mixture under preparation, consisting mainly of phase A. Then phases F and G were introduced, still without using a homogenizer. Phase H was added to adjust the pH to a value of approximately 6.

### Example 7-2

The formulation was based on the following ingredients:

| Phase | Ingredient according to INCI nomenclature | % by mass |
|---|---|---|
| A | Suspension of microcapsules according to the | 55 |
| | invention, at 60% | |
| A | Citric acid | 0.1 |
| B | Xanthan gum | 0.1 |
| B | Hydroxypropyl guar | 0.15 |
| C | Aqua | 8.85 |
| C | Disodium EDTA | 0.05 |
| D | Sodium stearoyl glutamate | 1 |
| E | Cetyl alcohol | 1 |
| E | Sucrose polystearate, Cetyl palmitate | 3 |
| E | Dicaprylyl carbonate, Tocopherol | 12 |
| E | Squalane | 5 |
| E | Butyrospermum Parkii Butter | 2 |
| E | Tricontanyl PVP | 2 |
| F | Undecane, Tridecane, Tocopherol | 8 |
| G | Tocopherol, Helianthus Annuus Seed Oil | 0.5 |
| H | Phenoxyethanol, Ethylhexylglycerin | 1 |
| I | Citric acid | 0.25 |

For this test a planetary stirrer was used, but no homogenizer. The components of phase A were cold mixed without using a homogenizer. Then the constituents of phase B were dispersed in phase A, still without using a homogenizer, then the ingredients of phase C, previously well dissolved, were added, and then phase D was added. Phase E was heated to 80° C. to mix its constituents well, then it was added to phase F (unheated), before introducing this mixture of phases E and F, still without using a homogenizer, into the mixture under preparation, consisting mainly of phase A. Then phases G and H were introduced, still without using a homogenizer. Phase I was added to adjust the pH to a value of approximately 6.

### Example 7-3

The formulation was based on the following ingredients:

| Phase | Ingredient according to INCI nomenclature | % by mass |
|---|---|---|
| A | Aqua | 53.65 |
| A | Disodium EDTA | 0.1 |
| A | Glycerin | 5 |
| B | Polyacrylate Crosspolymer-6 | 1.5 |
| C | Aqua, Acrylate copolymer | 2 |
| D | Suspension de microcapsules selon | 33 |
| | l'invention, à 99% | |
| E | Phenoxyethanol, Ethylhexylglycerin | 1 |
| E | Tocopherol, Helianthus Annuus Seed Oil | 0.5 |
| F | Dimethicone | 3 |
| G | Citric acid | 0.25 |

For this test a planetary mixer was used, but no homogenizer. The constituents of phase A were cold mixed, then the constituents of phase B were dispersed in phase A under vigorous stirring, and then phase C was added. Phase D was added to this mixture without using a homogenizer. Then the ingredients of phase E were mixed and phase E was introduced into the mixture under preparation, which was mainly made up of phase A, without using a homogenizer. Phase E was then slowly introduced under strong planetary agitation, but still without using a homogenizer. Phase I was added to adjust the pH to a value of approximately 6.

### Example 7-4

The formulation was based on the following ingredients:

| Phase | Ingredient according to INCI nomenclature | % by mass |
|---|---|---|
| A | Aqua | 22.65 |
| A | Disodium EDTA | 0.1 |
| B | Polyacrylate Crosspolymer-6 | 1.5 |
| C | Aqua, Acrylate copolymer | 2 |
| D | Suspension of microcapsules according to the | 66 |
| | invention, at 50% | |
| E | Phenoxyethanol, Ethylhexylglycerin | 1 |
| E | Tocopherol, Helianthus Annuus Seed Oil | 0.5 |
| F | Undecane, Tridecane, Tocopherol | 3 |
| F | Dimethicone | 3 |
| G | Citric acid | 0.25 |

The components of phase A were mixed cold, then the components of phase B were dispersed in phase A under vigorous stirring; for this the use of a homogenizer is advantageous. Under the same conditions, phase C was then added. The rest of the process was carried out without a homogenizer and with planetary stirring. Phase D was then introduced. The ingredients of phase E were then mixed and phase E was introduced into the mixture being prepared under planetary stirring, but without using a homogenizer.

Then the ingredients of phase E were mixed and phase E was introduced into the mixture under preparation, which was mainly made up of phase D, without using a homogenizer. Phase F was then slowly introduced with vigorous planetary stirring, but still without using a homogenizer. Phase G was added to adjust the pH to a value of approximately 6.

1-15. (canceled)

16. A core-shell type microcapsule, comprising:
a core containing at least one active ingredient; and
a shell forming a wall around the core and representing a mass fraction of at least 35% of the total mass of the microcapsule, the shell having at least one crosslinked polymer formed by interfacial polymerization.

17. The core-shell microcapsule of claim 16, wherein the at least one crosslinked polymer is chosen from polyurethane and/or polyurea.

18. The core-shell microcapsule of claim 16, wherein:
the shell comprises at least one anionic surfactant and/or at least one non-ionic surfactant, and
the at least one non-ionic surfactant is selected from a group formed by: polyethylene glycol esters, polyethylene glycol ethers, polyglycerol esters, esters of sorbitol derivatives such as sorbitan stearate, sucrose esters, and polysorbates.

19. The core-shell microcapsule of claim 16, wherein the core-shell microcapsules have a size an average size D_{S50} of less than 0.60 μm.

20. The core-shell microcapsule of claim 16, wherein the at least one active ingredient is selected from sunscreens.

21. The core-shell microcapsule of claim 16, wherein a plurality of core-shell microcapsules form a sun protection formulation or an anti-photoaging formulation.

22. The core-shell microcapsule of claim 21, wherein the sun protection formulation provides sun protection for skin or skin appendages and the anti-photoaging formulation provides protection against photo-aging of skin or skin appendages.

23. The core-shell microcapsule of claim 22, wherein the sun protection formulation or an anti-photoaging formulation comprises at least 35% by mass of core-shell microcapsules relative to a total weight of the sun protection formulation or an anti-photoaging formulation.

24. The core-shell microcapsule of claim 23, wherein the sun protection formulation has a sun protection factor SPF of at least 50.

25. The core-shell microcapsule of claim 21, wherein the sun protection formulation is for sun protection and the anti-photoaging formulation are formed as a liquid emulsion or a pasty emulsions, or formed as solid emulsions, or formed as non-emulsions.

26. A method for manufacturing, by interfacial polymerization, core-shell microcapsules that include a core containing at least one active ingredient, a shell forming a wall around the core and representing a mass fraction of at least 35% of the total mass of the microcapsule, the shell having at least one crosslinked polymer formed from at least two precursor that include a compound A and a compound B, the method comprising:
(a) mixing the at least one active ingredient, a solvent, and at least one compound A comprising more than two functional groups A′ to obtain a lipophilic phase;
(b) introducing, with stirring, the lipophilic phase obtained in step (a) into an aqueous continuous phase to form an emulsion;
(c) maintaining, with stirring, the emulsion at a temperature of between approximately 55° C. and approximately 65° C., and introducing, with stirring, an aqueous solution comprising at least one compound B comprising more than two functional groups B′ to the emulsion obtained at step (b) to react the functional groups A′ of the compound A comprising more than two functional groups A′ with the functional groups B′ of the compound B to form the crosslinked polymer shell.

27. The method of claim 26, wherein the stirring during step (b) is performed with a tangential speed greater than the stirring during the introduction of the aqueous solution comprising at least one compound B in step (c).

28. The method of claim 27, wherein the tangential speed is between 10 m/s and 30 m/s.

29. The method of claim 26, wherein:
the aqueous continuous phase comprises at least one anionic surfactant and/or one non-ionic surfactant, and
the anionic surfactant is chosen from magnesium lauryl ether sulfate and sodium lauryl ether sulfate.

30. The method of claim 26, wherein the NaCl content in the emulsion obtained in step (b) is between 0.15% and 0.90% by mass.

31. The method of claim 26, wherein the functional groups A′ of compound A are chosen from isocyanate groups.

32. The method of claim 31, wherein compound A is selected from a group formed by:
dicyclohexylmethane 4,4′-diisocyanate, hexamethylene 1,6-diisocyanate, isophorone diisocyanate, trimethylhexamethylene diisocyanate, hexamethylene 1,6-diisocyanate trimer, isophorone diisocyanate trimer, 1,4-cyclohexane diisocyanate, 1,4-(dimethylisocyanato) cyclohexane, hexamethylene diisocyanate biuret, hexamethylene diisocyanate biuret (CAS No. 4035-89-6), trimethylene diisocyanate, propylene-1,2-diisocyanate, butylene-1,2-diisocyanate, tetramethylene diisocyanate, pentamethylene diisocyanate, hexamethylene diisocyanate, 4-(isocyanatomethyl)-1,8-octyl diisocyanate;
mixtures between aliphatic diisocyanates and aliphatic triisocyanates,
aromatic polyisocyanates that include at least of 2,4-toluene diisocyanate, 2,6-toluene diisocyanate, naphthalene diisocyanate, diphenylmethane diisocyanate, triphenylmethane-p,p′,p″-trityl triisocyanate, and
aromatic isocyanates that include at least of toluene diisocyanate, polymethylene polyphenylisocyanate, 2,4,4′-diphenyl ether triisocyanate, polymethylene polyphenylisocyanate, 2,4,4′-diphenyl ether triisocyanate, 3,3′-dimethyl-4,4′-diphenyl diisocyanate, 3,3-dimethoxy-4,4′-diphenyl diisocyanate, 1,5-naphthalene diisocyanate, 4,4′,4″-triphenylmethane triisocyanate, isophoron diisocyanate.

33. The method of claim 32, wherein the functional groups B′ of compound B are chosen so that compound B is an amine.

34. The method of claim 33, wherein compound B is selected from a group formed by ethylene diamine, diethylene triamine, propylene diamine, tetraethylene pentaamine, pentamethylene hexamine, an alpha omega diamine, propylene-1,3-diamine, tetramethylene diamine, pentamethylene diamine, 1,6-hexamethylene diamine, triethylene triamine, pentaethylene hexamine, 1,3-phenylene diamine, 2,4-toluylene diamine, 4,4′-diaminodiphenyl methane, 1,5-diamino naphthalene, 1,3,5-triaminobenzene, 2,4,6-triaminotoluene, 1,3,6-triamino naphthalene, 2,4,4′-triaminodiphenyl ether, 3,4,5-triamino-1,2,4-triazole, bis(hexamethylene triamide), 1,4,5,8-tetraamino anthraquinone.

35. The method of claim 26, wherein the crosslinked polymer is chosen from polyurea or polyurethane.
